(19) United States
(12) Patent Application Publication
(10) Pub. No.: US 2018/0120276 A1
(43) Pub. Date: May 3, 2018

(54) GAS MOLECULE SENSOR

(71) Applicant: POLESTAR TECHNOLOGIES, INC., Needham Heights, MA (US)

(72) Inventors: James Kane, Norfolk, MA (US); Justin Harris, Chelmsford, MA (US); James Biagioni, Georgetown, MA (US); Timothy Bortz, Chester, NH (US)

(21) Appl. No.: 15/340,328

(22) Filed: Nov. 1, 2016

(51) Int. Cl. G01N 33/00; G01N 31/22; G01N 21/64; G01N 21/78; G01N 21/79; G01N 21/25

(52) U.S. Cl. CPC G01N 33/004; G01N 31/224; G01N 31/225; G01N 2201/062; G01N 21/783; G01N 21/79; G01N 21/251; G01N 21/6428

(57) ABSTRACT

The present disclosure relates to compositions, films and devices for sensing gas molecules. More particularly, the present disclosure relates to compositions, films and devices for sensing gas molecules utilizing optical sensing techniques having improved optical properties.

### STATEMENT REGARDING FEDERALLY SPONSORED RESEARCH OR DEVELOPMENT

(This invention was made partially with U.S. Government support from the U.S. Army Medical Research Acquisition Activity (USAMRAA) under SBIR contract #W81XH-10-C-0039. The federal government may nave certain rights in the invention.

### FIELD OF DISCLOSURE

The present disclosure relates to devices for sensing gas molecules. More particularly, the present disclosure relates to devices for sensing gas molecules utilizing optical sensing techniques having improved optical properties. Further, the active portion of the sensor is a film and is replaceable.

### BACKGROUND OF THE DISCLOSURE

Gas molecule sensors are known in the art. Many of these detectors use semiconductor-based technology, some of which measure the molecular ratio of designated gases by resistive methods. Other gas molecule sensors utilize liquid crystal technology wherein the morphology of the liquid crystal changes when it is exposed to certain gases. These devices use optical methods to determine the type and amount of gas that caused the change. Problems associated with this method include temperature and moisture dependence of the sensing ability as well as poor differentiation between like molecules. Still other devices are extremely complicate and bulky, such as those that require hermetic sealing, silicon wafers, a multitude of thin films, such as ceramic films and resistive thin films, and in some case refrigeration and high voltages.

Gas molecule sensors specific for carbon dioxide and/or oxygen are known in the literature. For example, WO 99/06821 discloses a fluorometric determination in which at least two luminescent materials are used. The materials are differentiated in that one has a short luminescent life time while the other has a longer luminescent lifetime. Both materials have similar spectral characteristics which limit the selection available for useful for the application. As well, the longer lifetime material is sintered into a glass to “immobilize” it.

An optical sensor for oxygen detection has been disclosed based on the fluorescent lifetime detection of a ruthenium dye. This method uses a time gated measurement. The film is separate from the light source (pulsed LED), a detector and a fiber optic reader pen. In this method, the film is places inside a food or beverage package; then the device containing the LED, detector and fiber pen are manually placed in contact to measure the lifetime difference. This process requires manual reading as well as two separate parts which complicates the process.

Carbon dioxides sensors that include films as the sensing material are known in the art. In such sensors, the presence of a particular gas such as carbon dioxide in air contacting the sensing film changes the optical properties of the film, such as, the color of light reflected off of the film changes. A light source, such as a light-emitting diode (LED), is shone through the film, while an optical detector on the other side of the film detects the color, or other, optical change. The accuracy and repeatability of these sensors are hindered by ambient air, ambient moisture and ambient air pressure.

Many gas molecule analyzers are large and not conducive to inclusion in masks, head gear uniforms, and the like. Many rely on mechanical components to direct the gas through the detection device, requiring large, and sometimes continuous, power sources.

Other sensors are based on redox chemistry which can be unreliable based on interactions with other ambient materials.

Thus, there is a need to improve the sensitivity, size, accuracy, weight and compatibility of such sensors. Moreover, there is a need to provide a sensor that has a mechanical construction which facilities not only ease of installation and use, but also ease of access to the sensing film so that the film can be quickly and conveniently replaced as needed.

It is accordingly with certain aspects of the current disclosure to provide novel, lightweight, uncomplicated, optically responsive gas molecule sensing devises that address the shortcomings of the currently described sensing devices.

### SUMMARY OF THE EXEMPLARY EMBODIMENTS

It is an object of the current disclosure to overcome the deficiencies commonly associated with the prior art as discussed above and provide devices and methods that improve sensing of gas molecules utilizing improved optical sensing techniques having improved optical properties. The active portion of the sensor of the current disclosure is a film construction and is removable and replaceable.

In a first embodiment, disclosed and claimed herein is a sensing device for detecting gas molecules including at least one LED light generating component, at least one color sensor, and at least one removably attached gas molecule sensing film, wherein the at least one LED component and the at least one color sensor are situated so that no light emitted from an LED component directly impinges onto a color sensor.

In a second embodiment, disclosed and claimed herein is a sensing device of the above embodiment wherein the at least one removably attached gas molecule sensing film is positioned so that the at least one LED component is capable of impinging light emitting from the at least one LED component onto and/or into the gas molecule sensing film, and wherein the color sensor is positioned to receive light given off from the gas molecule sensing film.

In a third embodiment, disclosed and claimed herein are sensing devices of any of the above embodiments, wherein the gas molecule sensing film comprises at least one of a gas molecule reacting composition, a substrate and an adhesive.

In a fourth embodiment, disclosed and claimed herein are sensing devices of any of the above embodiments, wherein the gas molecule reacting composition comprises at least one of a gas molecule reacting component which is capable of causing the composition to either change color when exposed to and reacted with a selected gas molecule or is capable of causing a change in the fluorescent properties of the composition when exposed to and reacted with a selected gas molecule.

In a fifth embodiment, disclosed and claimed herein are sensing devices of any of the above embodiments, wherein the gas molecule reacting composition further comprises at least one gas permeable host polymer and at least one base.

In a sixth embodiment, disclosed and claimed herein are sensing devices of any of the above embodiments, wherein the base is chosen from the alkali metal hydroxides, the alkaline earth metal hydroxide, the tetra-coordinated ammonium hydroxides, amines, and cyclic amines, and tetra-coordinated phosphorus hydroxides.

In a seventh embodiment, disclosed and claimed herein are sensing devices of any of the above embodiments, wherein the gas molecule reacting composition comprises at least one fluorescent component which is capable of reacting with a selected gas molecule to alter the fluorescence properties of the component when the LED light impinges onto and/or into the film which has been exposed to the selected gas molecules.

In an eighth embodiment, disclosed and claimed herein are sensing devices of any of the above embodiment further comprising at least one nonreactive colorant or fluorescent component.

In a tenth embodiment, disclosed and claimed herein are sensing devices of any of the above embodiments, wherein the selected gas is carbon dioxide and/or oxygen.

In an eleventh embodiment, disclosed and claimed herein are sensing devices of any of the above embodiments, further comprising an optically matched potting material positioned around and between, and in intimate contact with, the at least one LED component and the at least one color sensor, and in intimate contact with one surface of the removably attached gas molecule sensing film, wherein the potting material provides a continuous optical conduit between the LED component, the film and the color sensor, wherein the optically matched potting material essentially matches the refractive index of the layer of the gas molecule sensing film with which it is in intimate contact.

In a twelfth embodiment, disclosed and claimed herein are sensing devices of any of the above embodiments, wherein the at least one LED component is separated from the color sensor by an opaque component, wherein the opaque component is capable of preventing light from reaching the color sensor directly without first impinging onto and/or into the gas molecule sensing film.

In a further embodiment, disclosed and claimed herein are sensing devices of any of the above embodiments, comprising a protective cap removably attached to a main housing, the protective cap comprising a vent for allowing air to pass through the vent and coming into contact with a gas molecule sensing film, and from preventing light from passing through the vent; a color sensing construction for detecting selected gas molecules positioned within the main housing comprising: at least one LED light generating component, at least one color sensor, at least one removably attached gas molecule sensing film, wherein the at least one LED component and the at least one color sensor are situated so that no light emitted from an LED directly impinges onto a color sensor, and an optically matched potting material positioned around and between, and in intimate contact with, the at least one LED component and the at least one color sensor, and in intimate contact with one surface of the removably attached gas molecule sensing film, wherein the potting material provides a continuous optical conduit between the LED component, the film and the color sensor; and electrical components contained within the main housing; wherein the gas molecule sensing film is positioned within the main housing and between the color sensor and the protective cap.

In a further embodiment, disclosed and claimed herein are sensing devices of any of the above embodiments, wherein the gas molecule sensing film changes color when exposed to one or more of the selected molecules in the air that passed through the vent, and wherein, when the gas molecule sensing film changes color, the color sensor component detects the change in color and outputs an electrical signal based on the detected change in color of the film.

In a further embodiment, disclosed and claimed herein are sensing devices of any of the above embodiments, wherein the gas molecule sensing film comprises at least one fluorescent component which is capable of reacting with a selected gas molecule to alter the fluorescence properties of the component when the LED light impinges onto and/or into the film which has been exposed to the selected gas molecules, and when the LED impinges onto and/or into the film, the color sensor detects the alteration in the fluorescence and outputs an electrical signal based on the fluorescence alteration.

In a further embodiment, disclosed and claimed herein are sensing devices of any of the above embodiments, wherein the at least one LED component is separated from the color sensor by an opaque component, wherein the opaque component is capable of preventing light from reaching the color sensor directly without first impinging onto and/or into the gas molecule sensing film.

In a further embodiment, disclosed and claimed herein are sensing devices of any of the above embodiments, wherein the gas molecule sensing film further comprises one or more nonreactive colorant and/or fluorescent component, wherein the nonreactive colorant and/or fluorescent component provides a constant emission which the color sensor senses and outputs an electrical signal.

### BRIEF DESCRIPTION OF THE FIGURES

FIG. 1 is a perspective view of one embodiment of the sensor of the current disclosure sensor from the top;

FIG. 2 is a perspective view of the embodiment of FIG. 1 from the bottom;

FIG. 3 is a partially exploded view of the embodiment of FIG. 1 with the protective cap 12 removed and showing a printed circuit board in greater detail;

FIG. 4 is an exploded view of the embodiment of FIG. 1;

FIG. 5 is a close-up view of the printed circuit board of FIG. 3;

FIG. 6 is a side view of the embodiment of FIG. 1;

FIG. 7 is a cross-sectional view of the embodiment of FIG. 1 taken along line C-C of FIG. 6;

FIG. 8 is a further exploded view of the embodiment of FIG. 1 showing the sensing film 52 between the protective cap 12 and the annular top surface 26 of the body main body of the sensor;

FIG. 9 is a further exploded view of the embodiment of FIG. 1 showing the sensing film installed atop the annular ring 26 of the main body of the sensor; and

FIG. 10 is a cross-sectional view showing the one embodiment of the sensing film in greater detail.

### DETAILED DESCRIPTION OF THE DISCLOSURE

As used herein the term “light” refers to electromagnetic radiation of any wavelength or spectrum of wavelengths and is not restricted, including, for example, visible light, UV light, infrared light, white light, or combinations thereof.

As used herein the term “essentially” implies plus or minus 5% and is not meant to imply 100% absolute.

As used herein the term “opaque” refers to a minimum of 95% barrier to the LED output whether it is IR, visible or UV radiation.

As used herein the phrase “fluorescent properties” means emission intensity, emission wavelength and/or emission lifetime.

As used herein the phrase “optically-matched” means the two materials have essentially the same refractive index such that minimal optical effects occur, such as, for example, reflection and other effects detrimental, to light travel.

As used herein the term “dyes” includes the class of dyes as well as pigments.

Disclosed and claimed herein is a sensing device for detecting gas molecules including at least one LED component, at least one color sensor component, and at least one removably attached gas molecule sensing film, wherein the at least one LED component and the at least one color sensor are situated so that no light emitted from an LED component directly impinges onto a color sensor.

One or more LEDs that emit light may be included in the current sensing device. The LED may emit a narrow or a broad band of wavelengths. The LED may be constructed so that the light emitted is “white” light in that it includes most, some or essentially all, of the color wavelengths of the visible spectrum. Generally, by incorporating proper phosphors in the LED, emitted light combinations will mimic white light, as is well known in the industry.

More than one LED component may be present in the current sensing device. Each may emit the same or different wavelengths or wavelength bands. For example, one LED may emit white light, another may emit infrared and still another may emit blue light while another emits UV light. The choice of wavelength emission is selected based on color or fluorescent emission changing components of the gas molecule sensing film, the baseline color of the film, when appropriate, as well as the color sensor.

The device includes one or more color sensor components situated to receive electromagnetic radiation coming from the gas molecule sensing film. Color sensors are well known in the art and include, for example, 32-pixel, 3-color detectors, and may be designed for specific wavelength or wavelength band sensitivity.

The LED component and the color sensor components are situated so that light coming directly from the LED does not impinge on the color sensors. Thus there may be an opaque barrier between the LED and the color sensor. The barrier may include, for example, an electronic component such as a thermistor, a resistor, a capacitor or other electronic component, a thin opaque wall and/or the LED may be coated on the side nearest the sensor with an opaque coating, such as for example, a black epoxy. The interior of the device within which the LED and color sensor is located is non-reflective further ensuring minimal LED radiation impingement on the color sensor.

The device also includes gas molecule sensing films which are situated so that the LED can shine light onto the film and radiate light to be sensed by the color sensor.

The gas molecule sensing film either changes color when exposed to the target gas or the fluorescent properties of the film is altered. In some embodiments of the current disclosure, the sensing film contains basic components which can act as phase transfer ingredients of the film. When the target gas is carbon dioxide, the basic component reacts with the carbon dioxide generating an acid which can protonate a pH indicator dye causing a color change or color shift, via the following equation:

Q⁺D⁻.xH₂O+CO₂←→Q⁺HCO₃⁻.(x−1)H₂O+DH

In some embodiments, pH-sensitive fluorescent dyes may be used wherein the emission bands change or shift when the dye becomes protonated when the composition is exposed to carbon dioxide.

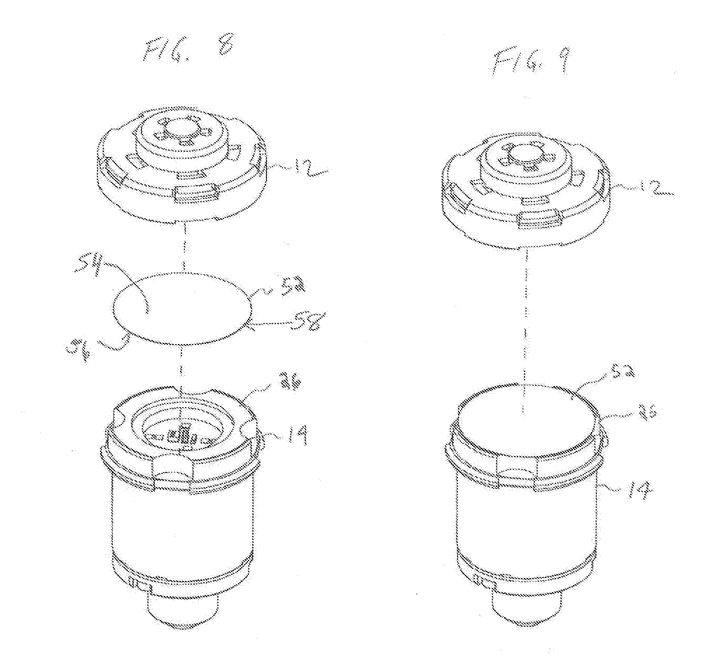

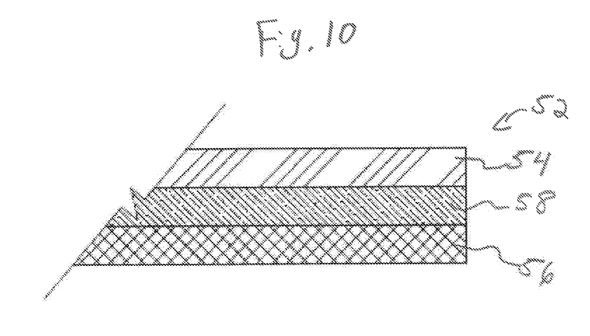

The film is situated so that light from the LED directly or indirectly impinges the film. The light may impinge on the surface or may penetrate a portion of the film or both. The light that then is reflected or emitted is received either directly or indirectly onto the color sensor components of the device. The reflected or emitted light may go through lens or other components to help place the light in condition for sensing by the color sensor. In one embodiment the one or more LED and one or more color sensor may be situated on the same plane while the gas molecule sensing film is situated parallel to the LED and sensor. See FIGS. 8 and 9.

In operation, the gas molecule sensing film is exposed to the target gas for which the film has been composed. The color or emission changing components of the film react with the gas causing the film to change color or alter its emission properties. Light from the LED impinges onto and/or into the film. Not to be held to color theory, the component of the film that has changed color due to exposure to the target gas will absorb certain known wavelengths of the light from the LED and reflect other known wavelengths of the light from the LED. The reflected wavelengths then impinge onto one or more color sensors. Electrical signals associated with the color sensing are sent from the color sensor to a microprocessor where the signal or signals are analyzed. In some embodiments the signals are compared to a predetermined non-color change baseline; and the color change is registered and the resulting information noted.

Target gasses for which the immediate disclosure is useful include, for example, carbon dioxide, carbon monoxide, oxygen, hydrogen cyanide, hydrogen sulfide, ammonia, oxides of nitrogen, halide gases such as chlorine, bromine, iodine and fluorine, and oxides of sulfur

The gas molecule sensing film may include a removable substrate, such as, for example, polyethylene terephthalate, polyethylene, polymethylmethacrylate and the like upon which the gas molecule reactive composition is coated. An adhesive may be applied to the surface of the gas molecule reactive composition such that the film adheres to a potting material, described infra. A cover sheet, such as, for example, polyethylene, polypropylene, polyolefin or other cover sheet material may be applied to the adhesive side of the construction. The cover sheet and substrate may have release coatings formulated to provide desired release characteristics, such as, for example, silicone release coating. The adhesive material may be formulated to match the refractive index of the potting material with which it comes into contact. In that matter there will be minimal light reflectance as the light from the LED travels through the potting material and the adhesive to the sensing film and back to the color sensor component. Suitable adhesives include, for example, acrylic based adhesives such as, for example, FLEXcon® V95, V98, V402, Franklin® Acrynax 11588, 3M® transfer adhesive 9877, rubber based adhesives, and the like.

Alternatively, the gas molecule reactive composition may be composed to have a desired amount of tack which would allow the composition, when coated and significantly dried, to adhere to the potting material without an index-matching adhesive.

The gas molecule reacting composition is composed of at least one of a gas molecule reacting component, at least one gas permeable host polymer and at least one base or phase transfer agent. The gas molecule reacting component is capable of either changing color when the composition is exposed to a selected gas molecule or is capable of causing an alteration in the fluorescent properties of the composition when the composition is exposed to and reacted with a selected gas molecule, as described above for carbon dioxide. Generally, in this embodiment the color changeable and fluorescent alterable materials are pH sensitive. So that when the gas reacts with the composition, for example, a base, the pH sensitive material is protonated and the color changes, or shifts, or the fluorescent properties are altered.

For carbon dioxide detection, suitable indicator dyes include, for example, metacresol purple, thymol blue, cresol red, phenol red, xylenol blue, a 3:1 mixture of cresol red and thymol blue, bromothymol blue, neutral red, phenolphthalein, thymolphthalein, malachite green, N,N-dimethylaniline, rosolic acid, alpha-naphtholphthalein, phenol red, bromocresol purple, bromocresol green, bromophenol red, p-nitrophenol, m-nitrophenol, curcumin, quinoline blue, thymolphthalein, 8-hydroxypyrene-1,3,6-trisulfonic acid trisodium salt and mixtures thereof. Any dye which changes color in response to a change in the local pH is suitable for the immediate disclosure.

Fluorescent materials suitable for the current disclosure include, for example, 1,3-dihydroxypyrene-6,8-disulfonate, fluorescein, umbelliferone, 4-methylumbelliferone, 3-benzothiazoylbelliferone, 7-hydroxycoumarin-3-carboxylic acid, 1-naphthol-2-sulphonate, 1-naphthol-4-sulfonate, 2-naphthol-6-sulphonate, 7-hydroxyflavone, 7-hydroxyisoflavone, 3-hydroxyxanthone, 3,6-dihydroxyxanthone, 7-hydroxy-4-methylchromon, 7-hydroxyepidone, 3-hydroxy-4-methylcoumarin, 8-hydroxypyrene-1, 3,6-trisulfonate, 3-hydroxyxacridone, salicylaldehyde semicarbazone, 2-hydroxycinnamic acid and the like. Any fluorescent compound with acidic hydroxyl groups capable for being deprotonated are suitable for the current disclosure.

The reactive colorants, fluorescent materials and/or the non-reactive colorants may be monomeric, oligomeric or polymeric.

The gas molecule reacting composition is also composed of at least one gas permeable host polymer. Suitable host polymers include, for example, polymethylpentene, polydimethylsiloxane, polystyrene, sol-gel glass polymer, poly(ethylene glycol) ethyl ether methacrylate, PVC, poly-styrene-co-pentafluorenestyrene, poly(isobutylmethacrylate-co-trifluoroethylmethacrylate), microporous polypropylene, polyvinyl butyral, cellulosic polymers, and the like, which allows the target gas to pass through to contact the color changing or emission altering composition.

Carbon dioxide reacting compositions are also composed of at least one base or phase transfer ingredients. Suitable bases and/or phase transfer agents include, for example, the tetraalkyl ammonium hydroxide, the tetraalkyl ammonium halides, the tetraalkyl ammonium sulfonates, sulfonates, sulfinates, p-toluenesulfonates and the like, tetraalkylphosphonium hydroxide. Herein alkyl means ethyl, propyl, butyl, pentyl, octyl and other hydrocarbons substituents. Other useful bases and/or phase transfer agents include the heterocyclic salt such as, for example, alkylpyridinium hydroxides and halides. Crown ether ionic complexes are also suitable for the current disclosure.

The gas molecule reactive composition may further include a drying agent as well as an optical dispersant such as, for example titanium dioxide, zinc oxide and the like.

Certain embodiments of the current disclosure include oxygen sensing film, compositions and devices. Not to be held theory but it is believed that oxygen quenches the luminescence of chosen indicator dyes, which include, for example fluorescent and phosphorescent dyes and pigments. Dyes useful for oxygen detection include, for example, the polycyclic aromatic hydrocarbons and transition metal complexes, such as, for example, pyrene-butanoic acid, tris(4,7-diphenyl-1,10-phenanthroline) ruthenium (II), tris-(4,7-diphenyl-1,10-phenanthroline) osmium (II), platinum, palladium and ruthenium octaethylporphorin, meso-tetra(pentafluorophenyl)porphine platinum (II) and the like. The oxygen reacting compositions may also include host polymers and other additives as described above for the carbon dioxide reacting compositions.

Certain embodiments of the current disclosure provide for gas molecule sensing films, compositions and devices that can detect more than one gas, such as, for example, both carbon dioxide and oxygen.

In certain other embodiments of the current disclosure, a protective coating may be applied to the surface of the dried gas molecule reactive composition. The coating may be gas permeable to allow the target gas to impinge onto or into the reactive composition. The protective layer may remain on the surface of the reactive composition while in operational use. The protective coating may be composed of any of a number of materials including, for example, fluorinated hydrocarbons, Teflon® materials polyolefins, low density polyethylene, porous versions of these materials like Gore-Tex® or Tyvek® and the like

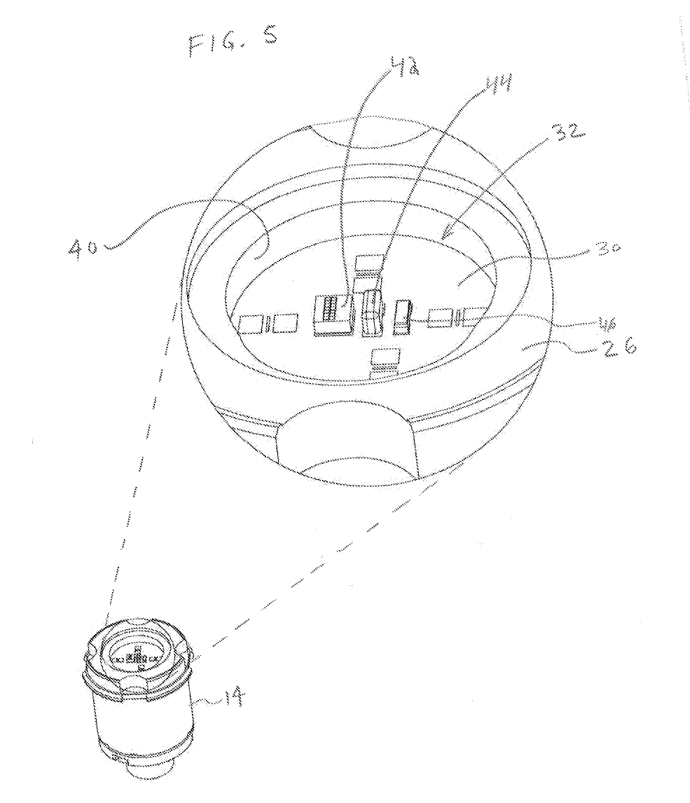

As described, the device contains one or more LEDs and one or more color sensors which can be separated by an opaque barrier. They can be situated on the same plane, for example, on a substrate or an electronic circuit board. Potting materials useful for the current disclosure are well known in the industry for such application as circuit boards and electronic microchips. It completely fills the space between the LED, the color sensor and the gas molecule sensing film and is essentially transparent to light emitted from the LED and light travelling from the film to the color sensor. It is either solid or gelatinous and resists shock, vibration, motion, gas permeation and corrosive agents. The potting material of the current disclosure is essentially refractive index matched with the surface of the gas molecule film which may include an adhesive or may be the coated gas molecule reactive composition. In this embodiment, light emitting from the LED and passing to the film is not impeded by reflections from the film's surface. These reflections are generally due to refractive index mismatches. Useful filler materials include thermosetting or photolytically cured silicones or epoxy compositions and are formulated to provide refractive index matching. Referring to FIG. 5, the filler material is applied into the cavity, or central aperture 32, formed by the circuit board 30, and the annular inner wall 40, essentially to the top of the cavity. The potting material is essentially bubble free and provides little to no interference for the light that travels through it. The continuous conduit between the components is essentially bubble free and provides little to no interference for the light that travel through it.

The gas molecule sensing film is positioned on the cured potting material either by an adhesive which is essentially optically-matched to the potting material, or the gas molecule reacting composition of the sensing film may be designed to contain enough tack to provide temporary adhesion of the film to the cured potting material.

The reactive composition of the gas molecule sensing film may include a colorant which is non-reactive to the target gas molecule. LED radiation may impinge on the nonreactive color and reflect light back to the color sensor. The nonreactive colorant does not change or shift its reflective spectrum or its fluorescent properties are unchanged when the film is exposed to the target radiation so that the reflected light may be analyzed and used as a baseline measurement to be compared with the reflection of emission from the colorants which have changes when exposed and reacted with the chosen target gas molecule. Many colorant, dyes and pigments fit this category and are well known in the industry. For example, in oxygen sensing films a combination of an oxygen insensitive luminophore, such as, for example, silicon octaethylporphine and an oxygen-sensitive luminophore, such as, for example, platinum octaethylporphine can be included the gas reacting composition. In this example dyes can be excited at 405 nm, but while the platinum complex has an oxygen-sensitive emission around 650 nm, the silicon complex's emission at 580 nm is essentially oxygen insensitive. A further example is the combination of the platinum group metal complexes such as oxygen-sensitive ruthenium tris(4,7-diphenyl-1,10-phenanthroline) and oxygen insensitive N,N′-bis(2,5-di-tert-butylphenyl)-3,4,9,10-perylenedicarboximide. Ratios of the oxygen-sensitive and -insensitive emissions could be generated based on the digital color sensor's ability to distinguish the two emissions

The gas molecule sensing film may include more than one coating layers. For example, a second coating may contain the non-reactive colorant which serves as a baseline measurement, or may include an addition gas molecule reacting composition.

The components of the gas molecule reactive composition include a dye, as described supra, from between about 0.10% and about 0.40%. The host polymer is between about 7.00% and about 15.00%, the base (transfer agent) is between about 1.00% and about 3.00%. When present, the optical dispersant is between about 1.00% and about 5.00%. A reference colorant, when present, is between about 0.05% and about 1.00%. (All percentages are shows in wt/wt % based on total weight of the component in a coating solution). The gas molecule reactive composition is coated from about a 14.7% solid in solvents such as, for example, methanol, ethanol, isopropanol, ethyl acetate, or other polar solvent, to give a dry thickness (dry being less than about 5% residual solvent) of between about 2.00 microns and about 8.00 microns. Any method known in the art for coating can be used to coat the composition, including, for example gravure coating, roller coating, reverse roller coating, wire bar coating, slot coating, and the like.

### EXAMPLES

### Example 1

12.3 g of Xylenol Blue was combined with a solution containing 2821 g ethanol, 319.3 g of polyvinylbutyral, 128 g rutile titanium dioxide, 71.5 g tetrabutylphosphonium hydroxide solution (40% by wt aqueous), and 97.7 g tributylphosphate. A 30 micron thick layer of this solution was deposited onto a 3 mil thick film of clear polyester using a 30 micron drawdown applicator bar and the ethanol evaporated at room temperature to produce a CO2 sensing film consisting of 54.5% polyvinylbutyral, 21.9% titanium dioxide, 16.6% tributylphosphate, 2.1% Xylenol Blue and 4.9% tetrabutyl phosphonium hydroxide. After evaporation of the ethanol a layer of 3M® transfer adhesive #9877 was applied to the uncoated side of the polyester substrate.

The CO² sensing film was adhered to the annular ring section of the sensing device, FIGS. 8 and 9, that included an epoxy potted printed circuit board on which were mounted a white light emitting diode (WLED) and digital color photodetector separated by a light barrier that prevented direct transmission of light from the WLED to the digital color photodetector, FIG. 7. The position of the adhered sensing film on the optical reader allowed light from the WLED to reflect off the sensing film and back to the digital color photodetector where the CO² dependent color of the film could be derived from a ratio of the signal levels detected with the red and blue pixels of the photodetector. The sensing film appeared deep blue in air and yielded the blue/red photodetector signal ratios shown in Table X upon exposure to various partial pressures of CO² at room temperature.

TABLE X

| PPCO² (mbar) | Blue/Red signal ratio |
|---|---|
| 0.0 | 0.526 |
| 2.3 | 0.670 |
| 4.5 | 0.753 |
| 6.8 | 0.855 |
| 9.9 | 0.937 |
| 13.6 | 1.020 |
| 19.9 | 1.123 |

### Example 2

12.3 g of Xylenol Blue was combined with a solution containing 2821 g ethanol, 319.3 g polyvinylbutyral, 71.5 g tetrabutylphosphonium hydroxide solution, and 97.7 g tributylphosphate. A 30 micron thick film of this solution was deposited onto a 3 mil thick film of clear polyester using a 30 micron drawdown applicator bar and the ethanol allowed to evaporate at room temperature. Following evaporation of the ethanol, a layer of porous Teflon (20 micron pore size) was applied to the top of the dry sensing film and a layer of 3M transfer adhesive #9877 was applied to the uncoated side of the polyester film. A patch of this film was adhered to the epoxy surface of the optical reader containing the WLED and digital color photodetector. The porous Teflon covered CO2 sensing film produced the blue/red photodetector signal ratio values in Table XX when exposed to various partial pressures of CO2 at room temperature.

TABLE XX

| PPCO2 (mbar) | Blue/Red signal ratio |
|---|---|
| 0 | 0.498 |
| 7.0 | 0.684 |
| 15.2 | 0.793 |
| 25.1 | 0.853 |
| 50.3 | 0.950 |

### Example 3

11.6 mg of tris(4,7-diphenyl-1,10-phenanthroline)ruthenium (II) chloride and 100 mg tributylphosphate were combined with 14.4 g of an ethanol solution containing 1.4 g polyvinylbutyral and 0.56 g rutile titanium dioxide. A 30 micron film of this solution was spread onto a 3 mil clear polyester film and the ethanol allowed to evaporate at room temperature leaving a 4 micron thick coating. A layer of 3M transfer adhesive #9877 was applied to the uncoated side of the polyester film after evaporation of the ethanol. An optical reader that included an epoxy potted printed circuit board equipped with a blue light emitting diode (BLED) and a digital color photodetector separated by a light barrier that prevented direct transmission of light from the BLED to the photodetector was used to measure the backscattered BLED light level and oxygen-dependent luminescent signal from the ruthenium complex while illuminating the film with the BLED. Values for the ratio of blue to green pixel signals from the photodetector under various partial pressures of oxygen at room temperature are provided in Table XXX

| PO2 (bar) | Blue/Green signal ratio |
|---|---|
| 0.00 | 6.02 |
| 0.35 | 8.21 |
| 0.70 | 10.28 |
| 1.00 | 11.88 |
| 1.53 | 14.91 |
| 2.00 | 16.89 |

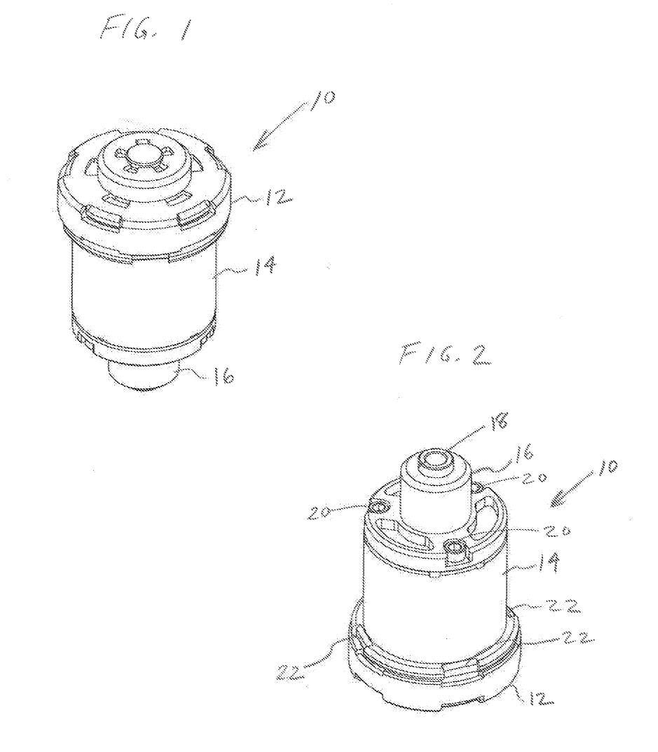

Turning now to the figures: FIG. 1 is a perspective view of one embodiment of the sensor 10 of the present disclosure from the top. The sensor 10 includes a protective cap 12, main housing 14 and connector housing 16. The protective cap 12 can be removably attached to the main housing 14 in any fashion known to those of ordinary skill in the art, such as a snap fit, a threaded connection, and the like. The sensor 10 can be installed in various pieces of equipment to sense gas molecular levels, such as, for example, in scuba diving equipment, medical equipment, airplanes, space vehicles, and the like.

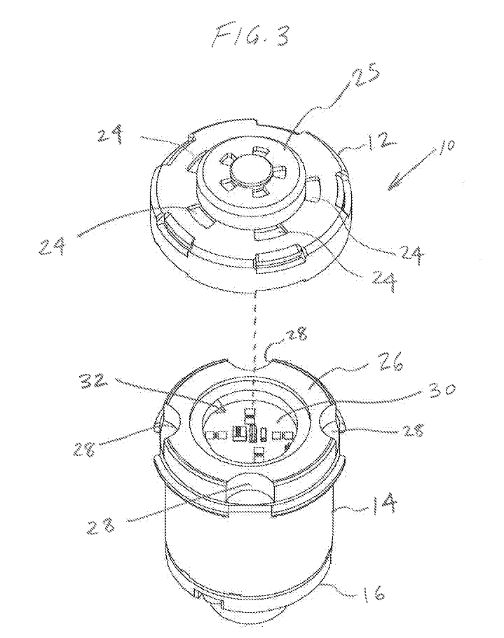

FIG. 2 is a perspective view of one embodiment of the sensor of the current disclosure 10, showing the bottom components of the sensor 10. The connector housing 16 includes a connector 18 for electrically connecting the sensor 10 to other equipment. The main housing 14 and connector housing 16 is attached showing a plurality of screws 20. The main housing 14 and connector housing 16 can also be attached through any means known by those skilled the art such as welding, gluing, bonding, and the like. Further, the main housing 14 and connector housing 16 can also be formed integrally with each other. The protective cap 12 includes a plurality of tabs 22 for assisting in the removal of the protective cap 12 from the main housing 14 in order to service and maintain the electrical components contained therein and/or to install, remove, or replace the sensing film as will be further discussed in detail below.

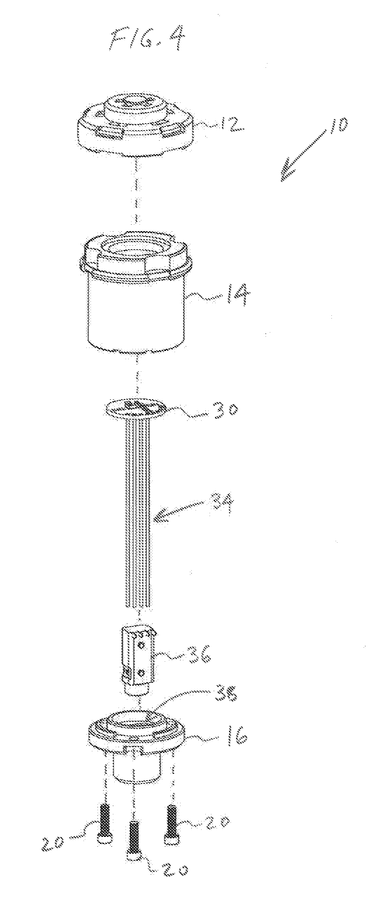

FIG. 3 is a partial exploded view of the sensor 10 embodiment of FIG. 1, showing the protective cap 12 removed and showing electrical components contained in the main housing 14. The protective cap 12 includes a plurality of vents 24 for allowing air to flow into the main housing 14, and preventing light from flowing into the main housing 14. The protective cap 12 also includes a center boss 25 for retaining a spring when the protective cap 12 is installed with the main housing 14. Any method known in the art could be used when installing the protective cap 12 to the main housing 14, such as using threads, screws, adhesives, and the like. The main housing 14 includes an annular top surface 26, scallops 28, and a printed circuit board 30. The annular top surface defines a central aperture 32 that receives the printed circuit board 30. The scallops 28 facilitate attachment and removal of a sensing film to the annular top surface 26.

FIG. 4 is an exploded view of the sensor 10 of the embodiment of FIG. 1, which shows further electrical components housed within the main housing 14. In particular, the printed circuit board 30 includes wiring 34, which is in electrical communication with the printed circuit board 30 and a connector body 36. The connector body 36 has a number of connectors corresponding to the number of wires 34 that are needed to supply power to, and electrical communication with, the printed circuit board 30. The connector housing 16 includes a central aperture 38 which receives the connector body 36. The connector body 36 could provide with power (+V_{DC} and ground) and data communications (TX/RX) signals to and from circuitry on the printed circuit board 30. The sensors of the current disclosure may include wireless communication configurations for communicating with devices, such as, for example, displays, readers, computers, and the like.

FIG. 5 is a close-up view of the annular top surface 26 showing the sensor 10 with the protective cap 12 removed. The annular top surface 26 includes an annular inner wall 40 which further defines central aperture 32. The annular inner wall 40 abuts the printed circuit board 30. The printed circuit board 30 further includes a color sensor 42, thermistor 44 and a light emitting diode (“LED”) 46. The LED 46 could emit white light. The thermistor 44 monitors temperature of the sensor 10. Additionally, the thermistor 44 is positioned between the color sensor 42 and the LED 46 so that it prevents light emitted from the LED 46 from directly impinging on the color sensor 42. Because of this, the color sensor 42 receives only light that is emitted from the LED 46 and reflected off of the sensing film, thereby increasing sensitivity of the sensor 10.

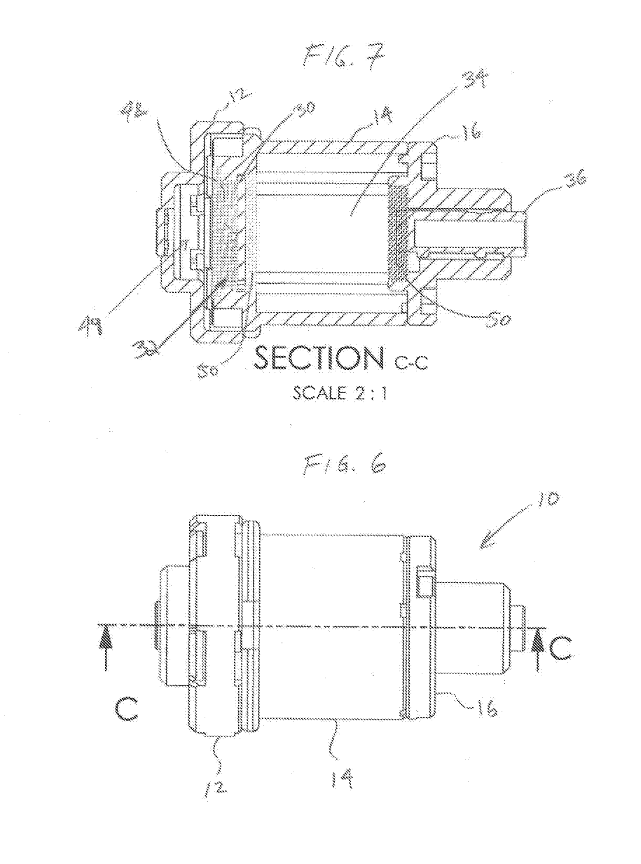

FIG. 6 is a side view of the sensor 10, and FIG. 7 is a cross-sectional view of sensor 10 taken along line C-C in FIG. 6. As shown in FIG. 7, the central aperture 32 is filled with an optical potting compound 48. The optical potting compound 48 can extend to the annular top surface 26. As discussed supra, the optical potting compound 48 has a refractive index that promotes proper signal transmission from the LED 46 to the sensing film, and from the sensing film to the color sensor 42. The protective cap 12 installed on the main housing 14 defines an air inflow region 49 which receives air, but not light, from the plurality of vents 24. Further shown in FIG. 7 are a plurality of compounds 50 which can be located at the connection between the printed circuit board 30 and the wiring 34, and also the connection between the electronics 34 and the connector body 36. The compounds 50 can isolate and seal electrical connections and provide a strong mechanical hold.

FIG. 8 is an exploded view of sensor 10, showing a sensing film 52. In this embodiment the sensing film 52 includes a top surface 54 and an optional bottom side adhesive 56, and an optional substrate 58. The sensing film 52 may be secured to the annular top surface 26 via the bottom side adhesive 56. It should be noted that the present disclosure includes any means known to those of ordinary skill in the art for securing the sensing film 52 to the annular top surface 26.

FIG. 9 is an exploded view of the sensor 10, showing the installation of the sensing film 52 to the annular top surface 26. The sensing film 52 can directly abut the top surface of the optical potting compound 48 to ensure proper signal transmission from the color sensor 42 to the sensing film 52. The sensing film 52 can be removed and serviced by removed the protective cap 12. The scallops 28 aid a person in installing, removing, and/or replacing the sensing film 52.

FIG. 10 is a cross-sectional view showing the sensing film 52 in greater detail. The top layer of the sensing film 54 includes one or more gas molecule reacting compound which changes the color of sensing film as described supra. The film may have an optional substrate 58 and an optional adhesive. The substrate 58 may provide structure to the sensing film 52 to facilitate installation, removal, and/or replacement. The substrate 58 is chosen to be essentially optically transparent to the light wavelengths of the emitted LED radiation. Also the substrate 58 is chosen to be essentially transparent to the radiation returning from the sensing film to the color detector.

The output of the sensor 10 and the monitoring thereof can be sent via an electronic communication signal which would originate at the printed circuit board 30, travel through the wires 34, and finally out through the central aperture 38 via any standard electrical connection line known to those of ordinary skill in the art. Alternatively, a wireless communication device could be added to the system to transmit the output from the sensor 10 to a smart phone, tablet, or any remote computer system used to monitor the output of sensor 10.

We claim:

1. A sensing device for detecting gas molecules comprising:
a. At least one LED light generating component,
b. At least one color sensor, and
c. At least one removably attached gas molecule sensing film,
wherein the at least one LED component and the at least one color sensor are situated so that no light emitted from an LED component directly impinges onto a color sensor.

2. The sensing device of claim 1, wherein the at least one removably attached gas molecule sensing film is positioned so that the at least one LED component is capable of impinging light emitting from the at least one LED component onto and/or into the gas molecule sensing film, and wherein the color sensor is positioned to receive light given off from the gas molecule sensing film.

3. The sensing device of claim 1, wherein the gas molecule sensing film comprises at least one of a gas molecule reacting composition, a substrate and an adhesive.

4. The sensing device of claim 3, wherein the gas molecule reacting composition comprises at least one of a gas molecule reacting component which is capable of causing the composition to either change color when exposed to and reacted with a selected gas molecule or is capable of causing a change in the fluorescent properties of the composition when exposed to and reacted with a selected gas molecule.

5. The sensing device of claim 4, wherein the gas molecule reacting composition further comprises at least one gas permeable host polymer and at least one base.

6. The sensing device of claim 5, wherein the base is chosen from the alkali metal hydroxides, the alkaline earth metal hydroxide, the tetra-coordinated ammonium hydroxides, amines, and cyclic amines, and tetra-coordinated phosphorus hydroxides.

7. The sensing device of claim 5, wherein the gas molecule reacting component is an indicator dye is chosen from a pH indicator, metacresol purple, thymol blue, cresol red, phenol red, xylenol blue, a 3:1 mixture of cresol red and thymol blue, bromothymol blue, neutral red, phenolphthalein, rosolic acid, alpha-naphtholphthalein, orange I, bromocresol purple, bromophenol red, p-nitrophenol, m-nitrophenol, curcumin, quinoline blue, thymolphthalein, 8-hydroxypyrene-1,3,6-trisulfonic acid trisodium salt and mixtures thereof.

8. The sensing device of claim 4, further comprising at least one nonreactive colorant or fluorescent component.

9. The sensing device of claim 3, wherein the gas molecule reacting composition comprises at least one fluorescent component which is capable of reacting with a selected gas molecule to alter the fluorescence properties of the component when the LED light impinges onto and/or into the film which has been exposed to the selected gas molecules.

10. The sensing device of claim 9, wherein the fluorescent component is chosen from a platinum group metal complex platinum group metal complexes of 1,10-phenanthroline and bipyrene derivatives including for example, ruthenium tris(4,7-diphenyl-1,10-phenanthroline) and osmium tris(4,4′-diphenyl-2,2′-bipyridine), metalloporphyrin complexes including platinum meso-tetra-perfluoroaromatic-porphorine, vanadyl octaethylporphine, palladium tetrakis(4-sulfonatophenyl)porphine, 1,3-dihydroxypyrene-6,8-disulfonate, fluorescein, umbelliferone, 4-methylumbelliferone, 3-benzothiazoylbelliferone, 7-hydroxycoumarin-3-carboxylic acid, 1-naphthol-2-sulphonate, 1-naphthol-4-sulfonate, 2-naphthol-6-sulphonate, 7-hydroxyflavone, 7-hydroxyisoflavone, 3-hydroxyxanthone, 3,6-dihydroxyxanthone, 7-hydroxy-4-methylchromon, 7-hydroxyepidone, 3-hydroxy-4-methylcoumarin, 8-hydroxy-1, 3,6-trisulfonate, 3-hydroxyxacridone, salicylaldehyde semicarbazone and 2-hydroxycinnamic acid

11. The sensing device of claim 9 further comprising at least one nonreactive colorant or fluorescent component.

12. The sensing device of claim 1, wherein the selected gas molecule is carbon dioxide and/or oxygen.

13. The sensing device of claim 1 further comprising an optically matched potting material positioned around and between, and in intimate contact with, the at least one LED component and the at least one color sensor, and in intimate contact with one surface of the removably attached gas molecule sensing film, wherein the potting material provides a continuous optical conduit between the LED component, the film and the color sensor.

14. The sensing device of claim 13, wherein the optically matched potting material essentially matches the refractive index of the layer of the gas molecule sensing film with which it is in intimate contact.

15. The sensing device of claim 1, wherein the at least one LED component is separated from the color sensor by an opaque component, wherein the opaque component is capable of preventing light from reaching the color sensor directly without first impinging onto and/or into the gas molecule sensing film.

16. A sensing device for detecting selected gas molecules comprising:
a. a protective cap removably attached to a main housing, the protective cap comprising a vent for allowing air to pass through the vent and coming into contact with a gas molecule sensing film, and from preventing light from passing through the vent;
b. a color sensing construction for detecting selected gas molecules positioned within the main housing comprising:
i. at least one LED light generating component,
ii. at least one color sensor,
iii. at least one removably attached gas molecule sensing film,
wherein the at least one LED component and the at least one color sensor are situated so that no light emitted from an LED directly impinges onto a color sensor, and
iv. an optically matched potting material positioned around and between, and in intimate contact with, the at least one LED component and the at least one color sensor, and in intimate contact with one surface of the removably attached gas molecule sensing film, wherein the potting material provides a continuous optical conduit between the LED component, the film and the color sensor; and
c. electrical components contained within the main housing;
wherein the gas molecule sensing film is positioned within the main housing and between the color sensor and the protective cap.

17. The sensing device of claim 16, wherein the gas molecule sensing film changes color when exposed to one or more of the selected molecules in the air that passed through the vent, and wherein, when the gas molecule sensing film changes color, the color sensor component detects the change in color and outputs an electrical signal based on the detected change in color of the film.

18. The sensing device of claim 16, wherein the gas molecule sensing film comprises at least one fluorescent component which is capable of reacting with a selected gas molecule to alter the fluorescence properties of the component when the LED light impinges onto and/or into the film which has been exposed to the selected gas molecules, and when the LED impinges onto and/or into the film, the color sensor detects the alteration in the fluorescence and outputs an electrical signal based on the fluorescence alteration.

19. The sensing device of claim 16, wherein the at least one LED component is separated from the color sensor by an opaque component, wherein the opaque component is capable of preventing light from reaching the color sensor directly without first impinging onto and/or into the gas molecule sensing film.

20. The sensing device of claim 16, wherein the gas molecule sensing film further comprises one or more nonreactive colorant and/or fluorescent component, wherein the nonreactive colorant and/or fluorescent component provides a constant emission which the color sensor senses and outputs an electrical signal.
